(19) United States
(12) Patent Application Publication
(10) Pub. No.: US 2006/0100173 A1
(43) Pub. Date: May 11, 2006

(54) Ophthalmic solution

(76) Inventors: Charles H. Powell, Irvine, CA (US); Stanley W. Huth, Newport Beach, CA (US)

Correspondence Address: Advanced Medical Optics, Inc.;Legal Department, 1700 E. St. Andrew Place, Santa Ana, CA, 92705, US

(21) Appl. No.: 11/271,448

(22) Filed: Nov. 9, 2005

Related U.S. Application Data
(60) Provisional application No. 60/626,371, filed on Nov. 9, 2004.

(51) Int. Cl. A61K 31/728; A61K 31/44

(52) U.S. Cl. 514/54; 514/358

(57) ABSTRACT

An improved ophthalmic composition comprising an anionic polymeric substance, such as Hyaluronic Acid and/or Carboxymethylcellulose, in combination with any of various cationic monomeric or dimeric antimicrobial agents, such as Cetylpyridinium Chloride and/or Alexidine Dihydrochloride, wherein said compositions provide additional comfort and biocompatibility with lenses without significantly affecting the antimicrobial efficacy of the antimicrobial agent and without therefore requiring a substantially increased concentration of the agent such as that could expose contact lens wearers to increased levels of the disinfecting agent. Solutions according to the present invention may be used for effective multipurpose contact lens disinfection compositions, lens packaging solution compositions, and/or eye drops such as rewetters and tears.

### BACKGROUND OF THE INVENTION

1. Field of the Invention

The invention relates to an improved ophthalmic composition wherein an anionic polymeric substance, especially the eye demulcents Hyaluronic Acid and/or Carboxymethylcellulose, is combined with a cationic monomeric or dimeric antimicrobial agent, wherein said compositions provide additional comfort and biocompatibility with lenses without significantly affecting the antimicrobial efficacy.

2. Description of the Related Art

Anionic polymers such as hyaluronic acid and carboxymethylcellulose have been noted for their moisturizing and lubricating properties in ophthalmic solutions. Such properties generally result in a reduction in irritation to the eye. Cationic antimicrobial agents have also been noted as beneficial to include in ophthalmic solutions. However, these two components are not viewed as compatible within one solution.

Cationic antimicrobial disinfecting agents have been shown to be compromised in their efficacy in the presence of certain anionic entities. For example, U.S. Pat. No. 5,858,346 teaches that poly[hexamethylene biguanide hydrochloride] (PHMB) and other non-oxidative disinfectants (typically cationic entities) are neutralized in their ability to damage cell walls, including cell walls of microorganisms, when combined with Carboxymethylcellulose (CMC) and/or various other negatively charged entities. Although the neutralization of the PHMB or other non-oxidative disinfectants alleviates irritation of the eye, the neutralization also results in loss of antimicrobial efficacy.

Similarly, U.S. Pat. No. 5,559,104, to Romeo et al., teaches the use of Cetylpyridinium Chloride (CPC) as an ion-pairing agent for the precipitation and purification of Hyaluronic Acid (HA) in a proposed manufacturing process for the HA biopolymer. Thus, that CPC would retain its antimicrobial activity in the presence of a large excess of Hyaluronic Acid or Carboxymethylcellulose is counterintuitive.

It would be advantageous to have compositions by which a decrease in irritability to the eye is not accompanied by a debilitating loss of antimicrobial efficacy. It is an object of the present invention to provide such compositions as well as methods of their use.

### DETAILED DESCRIPTION

A novel ophthalmic composition comprising a negatively charged polymeric substance and a cationic monomeric or dimeric antimicrobial agent is described, as well as its methods of use and preparation. Solutions according to the present invention may be used for effective multipurpose contact lens disinfection compositions, lens packaging solution compositions, and/or eye drops such as rewetters and tears. The composition provides additional comfort to the eye, and biocompatibility with lenses, without profoundly affecting the efficacy of the antimicrobial agent.

It has been discovered that certain naturally occurring and/or synthetic anionic polymeric substances previously presumed to be incompatible with cationic monomeric or dimeric antimicrobial agents do not profoundly inhibit the antimicrobial activity of said cationic antimicrobial agents.

In preferred embodiments, the compositions are compatible with typical and new additives to contact lens disinfection compositions, such as typical buffer systems, surfactants, demulcents, amino acids, and viscosity imparting agents, including hydroxypropylmethyl cellulose (HPMC).

In a preferred embodiment, the composition is comprised of specific concentration ranges of said cationic dimeric or monomeric antimicrobial agents and said water-soluble anionic polymeric substance.

In an alternative preferred embodiment, said anionic polymeric substance has a sufficiently low charge density such that the interaction between said anionic substance and said cationic monomeric or dimeric antimicrobial agent is sufficiently weak, and does not profoundly inhibit the microbiological activity of said antimicrobial agent.

In one embodiment, the composition comprises at least one anionic polymeric substance, such as Hyaluronic Acid and/or Carboxymethylcellulose, and at least one cationic monomeric or dimeric antimicrobial agent.

In another embodiment, the composition comprises at least one anionic polymeric substance and at least one cationic monomeric or dimeric antimicrobial agent, such as Cetylpyridinium Chloride, Alexidine Dihydrochloride, or other acceptable salts thereof.

In another embodiment, the composition preferably comprises at least one anionic polymeric substance and at least one cationic monomeric or dimeric antimicrobial agent, such as a monomeric biguanide-containing entity, such as Chlorhexidine and ophthalmically acceptable salts thereof, such as Chlorhexidine Hydrochloride, or a dimeric biguanide-containing entity, such as Alexidine and ophthalmically acceptable salts thereof, such as Alexidine Dihydrochloride. The salts of Alexidine and Chlorhexidine can be either organic or inorganic and are typically disinfecting gluconates, nitrates, acetates, phosphates, sulfates, halides and the like. In one embodiment, the composition comprises at least one anionic polymeric substance, such as Hyaluronic Acid and/or Carboxymethylcellulose, and at least one cationic monomeric or dimeric antimicrobial agent, such as a non-biguanide cationic monomeric antimicrobial agent, such as Cetylpyridinium Chloride.

In another embodiment, the composition comprises at least one anionic polymeric substance and at least one cationic monomeric or dimeric antimicrobial agent, such as Chiorhexidine or Alexidine; or a cationic monomeric agent, such as Cetylpyridinium Chloride, or ophthalmically acceptable salts thereof or mixtures thereof.

The aqueous medium of the present compositions typically includes a buffer component which is present in an amount effective to maintain the pH of the product in the desired range. The present compositions preferably include an effective amount of a tonicity adjusting component to provide the compositions with the desired tonicity.

The aqueous phase or component in the present compositions may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Boric acid may be combined with a geminal cis diol-containing substance, such as sorbitol, to effectively lower the pK of boric acid, thereby enhancing the buffer capacity of borate at the desirable physiologically optimal pH range. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄ 7H₂O and NaH₂PO₄ H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v % and more preferably varies from about 0.05 to about 0.5 w/v %.

The type and amount of buffer are selected so that the formulation meets the functional performance criteria of the composition, such as physicochemical attributes and shelf life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, more preferably about 6.5 to about 8.5 and still more preferably about 7.0 to about 8.0 is also acceptable. In one embodiment, the present composition has a pH of about 7.4.

The osmolarity of the present compositions may be adjusted with tonicity agents to a value which is compatible with the intended use of the compositions. For example, the osmolarity of the composition may be adjusted to approximate the osmotic pressure of normal tear fluid, which is equivalent to about 0.9 w/v % of sodium chloride in water. Examples of suitable tonicity adjusting agents include, without limitation: chloride salts of sodium, potassium, calcium and magnesium; dextrose; glycerin; propylene glycol; mannitol; sorbitol and the like; and mixtures thereof. In one embodiment, a combination of sodium chloride and potassium chloride are used to adjust the tonicity of the composition.

Tonicity agents are typically used in amounts ranging from about 0.001 to 2.5 w/v %. These amounts have been found to be useful in providing sufficient tonicity for maintaining ocular tissue integrity. Preferably, the tonicity agent(s) will be employed in an amount to provide a final osmotic value of 150 to 450 mOsm/kg, more preferably between about 220 to about 350 mOsm/kg and most preferably between about 270 to about 310 mOsm/kg.

In another embodiment, the composition preferably comprises at least one anionic polymeric substance, at least one cationic monomeric or dimeric antimicrobial agent and an aqueous medium comprising an alkali metal salt, such as sodium chloride, and a buffer agent, such as phosphoric acid and its related salts, boric acid and its related salts, or tri(2-hydroxymethyl)methylamine (Tris) and its related salts, such as to render the final osmolarity between 220 and 350 mOsm/kg and a preferred final pH between 6.5 and 8.5.

In another embodiment, the composition preferably comprises at least one anionic polymeric substance, at least one cationic monomeric or dimeric antimicrobial agent and an aqueous medium comprising one or more alkali metal salts, such as sodium chloride, and a buffer agent, such as phosphoric acid and its related salts, boric acid and its related salts, or tri(2-hydroxymethyl)methylamine (Tris) and its related salts, such as to render the final osmolarity between 280 and 310 mOsm/kg and a preferred final pH between 7.0 and 8.0.

In another embodiment, the composition preferably comprises at least one anionic polymeric substance, at least one cationic monomeric or dimeric antimicrobial agent and an aqueous medium comprising one or more surfactants, such as polyethylene oxide (PEO), polypropylene oxide (PPO), or block copolymers comprised of combinations of these, to achieve enhanced cleaning during lens treatment without significant loss of antimicrobial activity of the disinfecting agent(s). In an alternative embodiment, other PEO-PPO copolymer formats, wherein PEO-PPO chains are anchored to the functional groups of a poly-functional entity, such as the acetyl groups of ethylenediaminetetraacetic acid, as a means of increasing the molecular size of the surfactant, are used as the surfactant entity for said composition.

In another embodiment, the composition preferably comprises at least one anionic polymeric substance, at least one cationic monomeric or dimeric antimicrobial agent and an aqueous medium comprising one or more demulcents, such as propylene glycol, poly(hydroxypropylmethyl cellulose) or a short-chain polyethylene oxide, added preferably between from about as 0.05% to 1% w/v, to achieve enhanced cleaning and water retention of lenses, without significant loss of antimicrobial performance of the disinfecting agent(s).

In another embodiment, the composition preferably comprises at least one anionic polymeric substance, at least one cationic monomeric or dimeric antimicrobial agent and an aqueous medium comprising one or more chelating agents, such as ethylenediaminetetraacetic acid or one of its salts is added at an appropriate concentration, preferably from about 0.001% to about 0.01% w/v to insure solubilization of calcium from tear components during lens treatment with said contact lens disinfection composition.

In another embodiment, the composition preferably comprises at least one anionic polymeric substance, at least one cationic monomeric or dimeric antimicrobial agent and an aqueous medium comprising one or more amino acids, such as taurine, serine and glycine, added at a concentration to provide potential health benefit, preferably from about 0.001% to about 0.1% w/v, while not significantly altering the antimicrobial performance of the disinfecting agent(s).

In another embodiment, the composition preferably comprises at least one anionic polymeric substance, at least one cationic monomeric or dimeric antimicrobial agent and an aqueous medium comprising one or more vitamins or vitamin-related substances, such as tocopheryl polyethylene glycol succinate (TPGS) added at an appropriate level, preferably from about 0.0001% to about 0.1% w/v, to impart additional surfactant capacity to said disinfection composition.

In another embodiment, the composition preferably comprises at least one anionic polymeric substance, at least one cationic monomeric antimicrobial agent and an aqueous medium comprising one or more viscosity modifying agents or components, such as cellulose polymers, including hydroxypropylmethyl cellulose (HPMC), hydroxyethyl cellulose (HEC), ethyl hydroxyethyl cellulose, hydroxypropyl cellulose, methyl cellulose and carboxymethyl cellulose; carbomers (e.g. carbopol. RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

In another embodiment, the composition preferably comprises at least one anionic polymeric substance, at least one cationic monomeric or dimeric antimicrobial agent and an aqueous medium, wherein the anionic polymeric substance is hyaluronic acid with molecular weight preferably between 70,000 and 4 million daltons and more preferably a molecular weight between 700,000 and 2 million daltons.

In another embodiment, the composition preferably comprises at least one anionic polymeric substance, at least one cationic monomeric or dimeric antimicrobial agent and an aqueous medium, wherein the anionic polymeric substance is hyaluronic acid preferably added at a concentration between about 0.001% to about 1% w/v and more preferably added at a concentration between about 0.005% to about 0.2% w/v.

In another embodiment, the composition preferably comprises at least one anionic polymeric substance, at least one cationic monomeric or dimeric antimicrobial agent and an aqueous medium, wherein the anionic polymeric substance is hyaluronic acid with molecular weight preferably between 750,000 and 2 million daltons and is preferably added at a concentration between about 0.005% to about 0.2% w/v.

In another embodiment, the composition preferably comprises at least one anionic polymeric substance, at least one cationic monomeric or dimeric antimicrobial agent and an aqueous medium, wherein the cationic dimeric antimicrobial agent is Alexidine Dihydrochloride.

In another embodiment, the composition preferably comprises at least one anionic polymeric substance, at least one cationic monomeric or dimeric antimicrobial agent and an aqueous medium, wherein the cationic dimeric antimicrobial agent is Alexidine Dihydrochloride added at a concentration preferably between about 1 to about 5 ppm and more preferably between from about 1.0 or 1.5 to about 4.5 ppm.

The following concentration ranges are contemplated for the anionic polymeric substances considered herein: If the anionic polymeric substance is hyaluronic acid, the preferred concentration of said anionic polymeric substance, and depending on its molecular weight, is 0.001% to 1% w/v and, more preferably, 0.01% to 0.1%. If the anionic polymeric substance is carboxymethylcellulose, the preferred concentration of said anionic polymeric substance, and depending on its molecular weight, is 0.002% to 2% and, more preferably, 0.1% to 1.0%. One of ordinary skill in the art will be able to determine appropriate concentration ranges if a different anionic polymer is proposed for use.

The following concentration ranges are contemplated for the monomeric and dimeric cationic antimicrobial agents considered herein: The preferred concentration of said monomeric or dimeric antimicrobial agent is 0.2 grams per liter (ppm) to 20 ppm and, more preferably, 0.5 ppm to 4 ppm.

In another embodiment, the composition preferably comprises at least one anionic polymeric substance, at least one cationic monomeric or dimeric antimicrobial agent and an aqueous medium, wherein the cationic dimeric antimicrobial agent is Alexidine Dihydrochloride and the anionic polymeric substance is hyaluronic acid.

In another embodiment, the composition preferably comprises at least one anionic polymeric substance, at least one cationic monomeric or dimeric antimicrobial agent and an aqueous medium, wherein the anionic polymeric substance is hyaluronic acid.

In another embodiment, the composition preferably comprises at least one anionic polymeric substance, at least one cationic monomeric antimicrobial or dimeric agent and an aqueous medium, wherein the cationic monomeric antimicrobial agent is cetylpyridinium chloride.

In another embodiment, the composition preferably comprises at least one anionic polymeric substance, at least one cationic monomeric or dimeric antimicrobial agent and an aqueous medium, wherein the cationic monomeric antimicrobial agent is cetylpyridinium chloride and is preferably added at a concentration between about 0.5 to about 5 ppm, more preferably at a concentration of about 1 to about 3 ppm, and most preferably at a concentration of about 1.3 to about 2.3 ppm.

In another embodiment, the composition preferably comprises at least one anionic polymeric substance, at least one cationic monomeric or dimeric antimicrobial agent and an aqueous medium, wherein the cationic monomeric antimicrobial agent is cetylpyridinium chloride and is preferably added at a concentration between about 0.5 to about 5 ppm, more preferably at a concentration of about I to about 3.5 ppm, and most preferably at a concentration of about 1.3 to about 2.3 ppm, and wherein the anionic polymeric substance is Hyaluronic Acid.

Methods for treating a contact lens using the herein described compositions are included within the scope of the invention. In general, such methods comprise contacting a contact lens with such a composition at conditions effective to provide the desired treatment to the contact lens.

The contact lens can be contacted with the composition, often in the form of a liquid aqueous medium, by immersing the lens in the composition. During at least a portion of the contacting time period, the composition containing the contact lens can be agitated, for example, by shaking the container containing the composition and contact lens, to facilitate the contact lens treatment, for example, the removal of deposit material from the lens. Before or after such contacting step, in contact lens cleaning, the contact lens may be manually rubbed to remove further deposit material from the lens. The cleaning method can also include rinsing the lens prior to or after the contacting step and/or rinsing the lens substantially free of the composition prior to returning the lens to the wearer's eye.

It will be understood by those of skill in the art that numerous and various modifications can be made without departing from the spirit of the present invention. Therefore, it should be clearly understood that the following examples are illustrative only and are not intended to limit the scope of the present invention.

### EXAMPLES

### Example 1

An example of the ophthalmic composition according to the present invention was prepared as shown in Table 1. This composition, which surprisingly meets the disinfecting standards set forth for a ‘no rub’ solution, may provide additional comfort to the eye, and biocompatibility with lenses. It is noted that the anionic polymeric substance (in this example, HA), does not profoundly affect the efficacy of the antimicrobial agent.

As shown in Table 1 below, Cetylpyridinium Chloride (CPC) at various concentrations is found to be somewhat moderated in antimicrobial performance in the presence of 0.075% Hyaluronic Acid (HA) with average molecular weight (MW) of about 1.7 million (M) daltons, as compared to 0.2% HPMC, when using a Tris buffered contact lens disinfection composition. However, the slight lowering of activity is deemed minor with respect to the ability to achieve sufficient kill at low concentration for a robust disinfection system for contact lenses. Table 1 shows that CPC activity toward three bacteria and two fungi (C. Albicans and F. Salami) is lowered on average only by about one-half log in the sum microbial kill level of five common microbes, relative to the HPMC-containing formula. The loss of some activity toward S. aureus indicates only slight ion pairing between CPC and this relatively high molecular weight HA material. The fact that only slight ion pairing is seen is contrary to the teachings of the prior art.

TABLE 1
Antimicrobial activity toward five organisms versus CPC concentration in a Tris buffered
formulation containing either 0.2% HPMC or 0.075% HA (MW = 1.7 M daltons)

| Ingredient | HA Formulations | | | | | HPMC Formulations | | | | | Complete ® |
|---|---|---|---|---|---|---|---|---|---|---|---|
| CPC (ppm) | 1.00 | 1.25 | 1.50 | 1.75 | 2.00 | 1.00 | 1.25 | 1.50 | 1.75 | 2.00 | Lot 30616 |
| HA (1.7 M) (%) | 0.075 | 0.075 | 0.075 | 0.075 | 0.075 | | | | | | (Control) |
| HPMC (%) | | | | | | 0.2 | 0.2 | 0.2 | 0.2 | 0.2 | |
| Pluronic ® F-87 (%) | 0.05 | 0.05 | 0.05 | 0.05 | 0.05 | 0.05 | 0.05 | 0.05 | 0.05 | 0.05 | |
| NaCl (%) | 0.59 | 0.59 | 0.59 | 0.59 | 0.59 | 0.59 | 0.59 | 0.59 | 0.59 | 0.59 | |
| KCl (%) | 0.14 | 0.14 | 0.14 | 0.14 | 0.14 | 0.14 | 0.14 | 0.14 | 0.14 | 0.14 | |
| Propylene glycol (%) | 0.5 | 0.5 | 0.5 | 0.5 | 0.5 | 0.5 | 0.5 | 0.5 | 0.5 | 0.5 | |
| EDTA (%) | 0.01 | 0.01 | 0.01 | 0.01 | 0.01 | 0.01 | 0.01 | 0.01 | 0.01 | 0.01 | |
| Taurine (%) | 0.05 | 0.05 | 0.05 | 0.05 | 0.05 | 0.05 | 0.05 | 0.05 | 0.05 | 0.05 | |
| Tris HCl (%) | 0.055 | 0.055 | 0.055 | 0.055 | 0.055 | 0.055 | 0.055 | 0.055 | 0.055 | 0.055 | |
| Tris (%) | 0.021 | 0.021 | 0.021 | 0.021 | 0.021 | 0.021 | 0.021 | 0.021 | 0.021 | 0.021 | |
| Organism | Log Drop in Live Organisms | | | | | | | | | | |
| S. marcescens 13880 | 1.59 | 2.17 | 2.82 | 3.29 | 3.53 | 1.03 | 2.69 | 3.09 | 3.87 | 3.74 | 4.65 |
| S. aureus 6538 | 2.38 | 2.26 | 3.21 | 3.41 | 3.76 | 3.61 | 2.86 | 3.98 | 4.15 | 4.76 | 4.76 |
| P. aeruginosa 9027 | 4.58 | 4.58 | 4.58 | 4.58 | 4.58 | 4.28 | 4.58 | 4.58 | 4.58 | 4.58 | 4.58 |
| C. albicans 10231 | 1.20 | 1.87 | 2.36 | 2.56 | 3.15 | 1.43 | 2.16 | 2.31 | 3.14 | 2.23 | 1.57 |
| F. solani 36031 | 0.68 | 0.55 | 0.80 | 0.70 | 0.77 | 0.70 | 0.42 | 0.41 | 0.51 | 0.51 | 2.37 |
| Sum | 10.43 | 11.43 | 13.77 | 14.54 | 15.79 | 11.05 | 12.71 | 14.37 | 16.25 | 15.82 | 17.93 |

### Example 2

Further examples of ophthalmic compositions according to the present invention were prepared as shown in Tables 2A and 2B. Again, the cationic antimicrobial does not profoundly affect the efficacy of the antimicrobial agent.

Comparing Table 1 and Table 2B, Alexidine Dihydrochloride (ALX) is demonstrated to exhibit more antimicrobial efficacy (AME) activity on a weight-per-volume basis than CPC, when Tris buffer with 0.2% HPMC is used as the base formula. However, comparison of Table 1 and Table 2A indicates that, in the presence of HA, CPC retains better activity toward S. Aureus than ALX. Further comparison of Table 1 and Table 2A shows that borate buffered CPC containing 0.075% HA (MW=1.7M) exhibits much higher activity than Tris buffered CPC containing the same 0.075% HA. Although both Tris buffered and borate buffered “Placebos” (formulas containing all ingredients except the antimicrobial agent) show little or no AME activity, borate provides a demonstrable synergism with CPC that substantially boosts the overall AME activity. Comparing Table 2A and Table 2B shows that the AME activity of ALX in Tris buffer is also moderated when 0.05% HA (MW=1.7M) is used as the demulcent instead of 0.2% HPMC. Thus, both CPC and ALX exhibit slight reduction in activity when HA having a MW of 1.7M is employed as the demulcent. The loss of only some activity again indicates only slight ion pairing between CPC and this relatively high molecular weight HA material. The fact that only slight ion pairing is seen is contrary to the teachings of the prior art.

TABLE 2A
Antimicrobial activity toward five organisms in HA-containing Tris and Borate buffered systems, respectively, versus ALX or CPC concentration

| Ingredient | ALX Formulations | | | | | CPC Formultaions | | | | | | Complete ® |
|---|---|---|---|---|---|---|---|---|---|---|---|---|
| ALX (ppm) | 1.25 | 1.50 | 1.75 | 2.00 | 2.50 | | | | | | | Exp: Sep. 05 |
| CPC (ppm) | | | | | | 1.00 | 1.50 | 1.70 | 1.85 | 2.00 | 2.15 | Lot 27321 |
| HA (M.W. = 1.7 M) (%) | 0.05 | 0.05 | 0.05 | 0.05 | 0.05 | 0.075 | 0.075 | 0.075 | 0.075 | 0.075 | 0.075 | (Control) |
| Tris HCl (%) | 0.055 | 0.055 | 0.055 | 0.055 | 0.055 | | | | | | | |
| Tris Base (%) | 0.021 | 0.021 | 0.021 | 0.021 | 0.021 | | | | | | | |
| NaCl (%) | 0.59 | 0.59 | 0.59 | 0.59 | 0.59 | 0.59 | 0.59 | 0.59 | 0.59 | 0.59 | 0.59 | |
| KCl (%) | 0.14 | 0.14 | 0.14 | 0.14 | 0.14 | 0.14 | 0.14 | 0.14 | 0.14 | 0.14 | 0.14 | |
| EDTA (%) | 0.01 | 0.01 | 0.01 | 0.01 | 0.01 | 0.01 | 0.01 | 0.01 | 0.01 | 0.01 | 0.01 | |
| Taurine (%) | 0.05 | 0.05 | 0.05 | 0.05 | 0.05 | 0.05 | 0.05 | 0.05 | 0.05 | 0.05 | 0.05 | |
| Pluronic ® F87 (%) | 0.05 | 0.05 | 0.05 | 0.05 | 0.05 | 0.05 | 0.05 | 0.05 | 0.05 | 0.05 | 0.05 | |
| Propylene Glycol (%) | 0.5 | 0.5 | 0.5 | 0.5 | 0.5 | | | | | | | |
| PEG 400 (%) | | | | | | 0.1 | 0.1 | 0.1 | 0.1 | 0.1 | 0.1 | |
| Boric acid (%) | | | | | | 0.6 | 0.6 | 0.6 | 0.6 | 0.6 | 0.6 | |
| NaOH, 1 N (%) | | | | | | 0.675 | 0.675 | 0.675 | 0.675 | 0.675 | 0.675 | |
| Organism | Log dop in viable organisms at 6 hours after ˜1 × E5 innoculum of specified organism: | | | | | | | | | | | |
| S. marcescens 13880 | 3.15 | 3.43 | 4.60 | 4.60 | 4.60 | 3.00 | 3.14 | 3.32 | 3.01 | 4.60 | 3.70 | 4.60 |
| S. marcescens (#2) | 3.59 | 4.59 | 4.59 | 4.59 | 4.59 | 1.46 | 4.59 | 4.59 | 4.59 | 3.29 | 3.29 | 3.29 |
| SM average | 3.37 | 4.01 | 4.60 | 4.60 | 4.60 | 2.23 | 3.87 | 3.96 | 3.80 | 3.95 | 3.50 | 3.95 |
| S. aureus 6538 | 1.53 | 2.59 | 3.07 | 3.16 | 3.21 | 3.01 | 2.40 | 3.54 | 3.00 | 3.94 | 4.15 | 4.85 |
| S. aureus (#2) | 1.29 | 1.95 | 2.67 | 3.04 | 3.68 | 2.19 | 4.05 | 3.65 | 3.70 | 4.95 | 4.05 | 4.95 |
| SA average | 1.41 | 2.27 | 2.87 | 3.10 | 3.45 | 2.60 | 3.23 | 3.60 | 3.35 | 4.45 | 4.10 | 4.90 |
| P. aeruginosa 9027 | 4.59 | 4.59 | 4.59 | 4.59 | 4.59 | 4.59 | 4.59 | 4.59 | 4.59 | 4.59 | 4.59 | 4.59 |
| C. albicans 10231 | 0.88 | 0.89 | 1.31 | 1.47 | 1.79 | 1.73 | 2.61 | 2.26 | 2.86 | 3.55 | 2.91 | 1.42 |
| F. solani 36031 | 1.93 | 3.14 | 3.71 | 4.19 | 3.58 | 2.19 | 3.11 | 3.07 | 4.19 | 3.58 | 3.41 | 3.07 |
| Sum of Organisms | 12.18 | 14.90 | 17.07 | 17.94 | 18.00 | 13.34 | 17.40 | 17.47 | 18.79 | 20.11 | 18.50 | 17.93 |

TABLE 2B
Antimicrobial activity in Tris buffered formulations
containing 0.2% HPMC toward five organisms versus
Alexidine Dihydrochloride concentration

| Formulation | HPMC Formulations | | | Complete ® |
|---|---|---|---|---|
| ALX (ppm) | 1.50 | 2.00 | 2.50 | Lot 27321 |
| HPMC (%) | 0.20 | 0.20 | 0.20 | Exp: Sep. 05 |
| Pluronic ® F-87 (%) | 0.05 | 0.05 | 0.05 | (Control) |
| NaCl (%) | 0.59 | 0.59 | 0.59 | |
| KCl (%) | 0.14 | 0.14 | 0.14 | |
| Propylene glycol (%) | 0.50 | 0.50 | 0.50 | |
| EDTA (%) | 0.01 | 0.01 | 0.01 | |
| Taurine (%) | 0.05 | 0.05 | 0.05 | |
| Tris HCl (%) | 0.055 | 0.055 | 0.055 | |
| Tris Base (%) | 0.021 | 0.021 | 0.021 | |
| Organism | Log drop in live organisms @ 6 hrs. | | | |
| S. marcescens 13880 | 4.83 | 4.83 | 3.75 | 3.53 |
| S. aureus 6538 | 3.69 | 4.51 | 4.81 | 4.51 |
| P. aeruginosa 9027 | 4.73 | 4.73 | 4.73 | 4.73 |
| C. albicans 10231 | 1.18 | 1.72 | 1.87 | 1.49 |
| F. solani 36031 | 2.18 | 2.73 | 4.18 | 2.97 |
| Sum | 16.61 | 18.52 | 19.34 | 17.23 |

### Example 3

Tests were performed to determine whether the molecular weight of the particular anionic polymer had any effect on the efficacy of the solution. Table 3 shows the effect of HA molecular weight upon the antimicrobial activity of three disinfection chemical entities: PHMB, ALX and CPC. Complete® Moisture Plus™ multi-purpose solution (containing 0.15% HPMC and 1.0 ppm PHMB) serves as the non-HA control for the PHMB-HA formulation, while the CPC non-HA control is presented in Table 3 (at 2.0 ppm CPC and 0.15% HPMC). The ALX non-HA control for 2.0 ppm ALX is found in Table 2B. The data demonstrate that HA having a molecular weight of 1.5M or 0.8M has no measurable reductive effect on the antimicrobial efficacy of these three chemical entities, relative to HPMC at 0.15% or 0.2%. The relatively high molecular weight version of HA, 1.7M, is seen to produce the greatest inhibition of antimicrobial efficacy toward PHMB, where the sum of the kill level is reduced by about 4 log relative to the non-HA containing control and the lower molecular weight HA PHMB compositions. The ALX compositions are also significantly affected in antimicrobial efficacy by the highest presented molecular weight version of HA (1.7M), loosing about 2 log of activity relative to the lower molecular weight entities. The CPC shows the least effect on efficacy by the presence of the largest tested HA (1.7M), losing less than 1 log of sum activity. The loss of only some activity again indicates only slight ion pairing between CPC and this relatively high molecular weight HA material. The fact that only slight ion pairing is seen is contrary to the teachings of the prior art.

TABLE 3
Antimicrobial activity of three different disinfection chemical entities in Tris buffered
formulations containing HA of three different molecular weights: 0.8 M, 1.5 M and 1.7 M

| Ingredient | CPC Formulations | | | | | | | Alex. Formulations | | |
|---|---|---|---|---|---|---|---|---|---|---|
| PHMB (ppm) | | | | | | | | | | |
| Alexidine (ppm) | | | | | | | | 2.0 | 2.0 | 2.0 |
| CPC (ppm) | 1.5 | 1.5 | 1.5 | 2.0 | 2.0 | 2.0 | 2.0 | | | |
| HA (MW = 0.8 M) (%) | 0.05 | | | 0.05 | | | | 0.05 | | |
| HA (MW = 1.5 M) (%) | | 0.05 | | | 0.05 | | | | 0.05 | |
| HA (MW = 1.7 M) (%) | | | 0.05 | | | 0.05 | | | | 0.05 |
| HPMC (%) | | | | | | | 0.15 | | | |
| Tris HCl (%) | 0.055 | 0.055 | 0.055 | 0.055 | 0.055 | 0.055 | 0.055 | 0.055 | 0.055 | 0.055 |
| Tris Base (%) | 0.021 | 0.021 | 0.021 | 0.021 | 0.021 | 0.021 | 0.021 | 0.021 | 0.021 | 0.021 |
| NaCl (%) | 0.59 | 0.59 | 0.59 | 0.59 | 0.59 | 0.59 | 0.59 | 0.59 | 0.59 | 0.59 |
| KCl (%) | 0.14 | 0.14 | 0.14 | 0.14 | 0.14 | 0.14 | 0.14 | 0.14 | 0.14 | 0.14 |
| Edetate Disodium (%) | 0.01 | 0.01 | 0.01 | 0.01 | 0.01 | 0.01 | 0.01 | 0.01 | 0.01 | 0.01 |
| Taurine (%) | 0.05 | 0.05 | 0.05 | 0.05 | 0.05 | 0.05 | 0.05 | 0.05 | 0.05 | 0.05 |
| Pluronic ® F87 (%) | 0.05 | 0.05 | 0.05 | 0.05 | 0.05 | 0.05 | 0.05 | 0.05 | 0.05 | 0.05 |
| Propylene Glycol | 0.5 | 0.5 | 0.5 | 0.5 | 0.5 | 0.5 | 0.5 | 0.5 | 0.5 | 0.5 |
| Organism | Log dop in viable organisms at 6 hours after ˜1 × E5 innoculum of specified organism: | | | | | | | | | |
| S. marcescens 13880 | 2.74 | 3.25 | 3.67 | 3.67 | 3.7 | 4.10 | 4.03 | 4.88 | 4.88 | 4.88 |
| S. aureus 6538 | 4.2 | 4.68 | 3.2 | 4.2 | 4.68 | 3.98 | 3.57 | 4.38 | 3.78 | 3.30 |
| P. aeruginosa 9027 | 4.56 | 4.56 | 4.56 | 4.56 | 3.71 | 4.56 | 4.56 | 4.56 | 4.56 | 4.56 |
| C. albicans 10231 | 2.87 | 3.24 | 2.94 | 3.10 | 2.85 | 2.8 | 3.39 | 1.6 | 1.86 | 1.74 |
| F. solani 36031 | 1.89 | 1.76 | 1.64 | 2.50 | 2.60 | 2.44 | 1.82 | 3.75 | 3.87 | 2.87 |
| Sum | 16.26 | 17.49 | 16.01 | 18.03 | 17.54 | 17.88 | 17.37 | 19.17 | 18.95 | 17.35 |

| Ingredient | PHMB Formulations | | | Complete ® |
|---|---|---|---|---|
| PHMB (ppm) | 1.0 | 1.0 | 1.0 | Lot 27321 |
| Alexidine (ppm) | | | | Exp: September 2005 |
| CPC (ppm) | | | | (Control) |
| HA (MW = 0.8 M) (%) | 0.05 | | | |
| HA (MW = 1.5 M) (%) | | 0.05 | | |
| HA (MW = 1.7 M) (%) | | | 0.05 | |
| HPMC (%) | | | | |
| Tris HCl (%) | 0.055 | 0.055 | 0.055 | |
| Tris Base (%) | 0.021 | 0.021 | 0.021 | |
| NaCl (%) | 0.59 | 0.59 | 0.59 | |
| KCl (%) | 0.14 | 0.14 | 0.14 | |
| Edetate Disodium (%) | 0.01 | 0.01 | 0.01 | |
| Taurine (%) | 0.05 | 0.05 | 0.05 | |
| Pluronic ® F87 (%) | 0.05 | 0.05 | 0.05 | |
| Propylene Glycol | 0.5 | 0.5 | 0.5 | |
| Organism | Log dop in viable organisms at 6 hours after ˜1 × E5 innoculum of specified organism: | | | |
| S. marcescens 13880 | 4.88 | 4.88 | 3.64 | 4.88 |
| S. aureus 6538 | 4.68 | 4.68 | 3.04 | 4.68 |
| P. aeruginosa 9027 | 4.56 | 4.56 | 4.56 | 4.56 |
| C. albicans 10231 | 1.35 | 1.52 | 0.46 | 1.83 |
| F. solani 36031 | 2.79 | 2.87 | 2.54 | 2.93 |
| Sum | 18.26 | 18.51 | 14.24 | 18.88 |

### Example 4

The following example shows the effect on antimicrobial activity of a monomeric antimicrobial agent, CPC, of the addition of 0.5% of a commercially obtained (from Hercules Inc., Wilmington, Del.) low viscosity form of carboxymethylcellulose. The resulting Test solution (Solution #4 in Table 4) exhibited a viscosity of 3.5 centipoises. The activity against three key organisms of the Test solution was evaluated in a series of increasing concentrations of CPC in solutions containing 0.2% HPMC instead of 0.5% CMC. The Test solution exhibited slightly higher activity against S. marcescens than the comparison solution having a slightly higher (but comparable) level of CPC (#5). The Test solution also meets the stand-alone criteria at this concentration of CPC. The Test and Control solutions only differ in the demulcent identity (CMC vs. HPMC) and concentration of CPC. The CPC level in the Test solution is bracketed by the CPC levels in Solutions #3 and #5. While the S. aureus log reduction is lower for the Test solution than for the bracketing concentrations of CPC control solutions, this degree of inhibition of activity against this organism is not prohibitive for the present applications.

TABLE 4
Relative antimicrobial activity of a Test solution (#4) containing
CPC in combination with low-viscosity CPC in a series of increasing
concentrations of CPC in combination with HPMC

| | Solution # | | | | |
|---|---|---|---|---|---|
| Ingredient | 1 | 2 | 3 | 4 | 5 |
| NaCl (%) | 0.55 | 0.55 | 0.55 | 0.55 | 0.55 |
| KCl (%) | 0.14 | 0.14 | 0.14 | 0.14 | 0.14 |
| PF-87 (%) | 0.05 | 0.05 | 0.05 | 0.55 | 0.55 |
| Taurine (%) | 0.05 | 0.05 | 0.05 | 0.05 | 0.05 |
| PEG-400 (%) | 0.10 | 0.10 | 0.10 | 0.10 | 0.10 |
| Sorbitol (%) | 0.20 | 0.20 | 0.20 | 0.20 | 0.20 |
| Boric Acid (%) | 0.10 | 0.10 | 0.10 | 0.10 | 0.10 |
| EDTA (%) | 0.01 | 0.01 | 0.01 | 0.01 | 0.01 |
| HPMC (%) | 0.20 | 0.20 | 0.20 | 0 | 0.20 |
| CMC (Low-Vis) (%) | 0 | 0 | 0 | 0.50 | 0.00 |
| CPC (ppm) | 0.595 | 0.809 | 1.03 | 1.25 | 1.35 |
| Organism | Log drop @ 6 hours | | | | |
| S. marcescens 13880 | 0.81 | 1.68 | 2.34 | 3.40 | 3.24 |
| S. aureus 6538 | 0.47 | 2.51 | 5.10 | 3.59 | 5.18 |
| F. solani 36031 | 0.98 | 1.36 | 1.66 | 1.92 | 2.22 |
| Sum | 2.26 | 5.55 | 9.10 | 8.91 | 10.64 |

What is claimed:

1. An ophthalmic composition comprising an anionic polymer and a monomeric or dimeric cationic antimicrobial agent.

2. The ophthalmic composition as in claim 1, wherein the anionic polymer is selected from the group consisting of carboxymethylcellulose, hyaluronic acid, hyaluronate, and mixtures thereof.

3. The ophthalmic composition as in claim 1, wherein the anionic polymer is present in an amount ranging from about 0.001 to about 1% w/v.

4. The ophthalmic composition as in claim 1, wherein the anionic polymer is present in an amount ranging from about 0.002 to about 2% w/v.

5. The ophthalmic composition as in claim 1, wherein the anionic polymer has a molecular weight of from about 70,000 to about 4 million Daltons.

6. The ophthalmic composition as in claim 1, wherein the antimicrobial agent cetylpyridinium chloride.

7. The ophthalmic composition as in claim 1, wherein the antimicrobial agent is present in an amount ranging from about 0.2 ppm to about 20 ppm.

8. The ophthalmic composition as in claim 1, further including a buffer in an amount ranging from about 0.01 to about 2.5% w/v.

9. The ophthalmic composition as in claim 1, further including a tonicity agent in an amount ranging from about 0.001 to about 2.5% w/v.

10. The ophthalmic composition as in claim 1, further including a demulcent in an amount ranging from about 0.05 to about 1% w/v.

11. The ophthalmic composition as in claim 1, further including a chelating agent in an amount ranging from about 0.001 to about 0.01% w/v.

12. The ophthalmic composition as in claim 1, further including a viscosity modifying agent in an amount ranging from about 0.01 to about 5% w/v.

13. An ophthalmic composition, wherein the solution comprises:
from about 0.001% to about 1% w/v hyaluronic acid, from about 0.2 to 20 ppm of a monomeric or dimeric cationic antimicrobial agent, and a buffer.

14. The ophthalmic composition as in claim 13, wherein the hyaluronic acid has a molecular weight of from about 70,000 to about 4 million Daltons.

15. The ophthalmic composition as in claim 13, wherein the antimicrobial agent cetylpyridinium chloride.

16. The ophthalmic composition as in claim 13, wherein the antimicrobial agent is present in an amount ranging from about 0.2 ppm to about 20 ppm.

17. The ophthalmic composition as in claim 13, further including a tonicity agent in an amount ranging from about 0.001 to about 2.5% w/v.

18. The ophthalmic composition as in claim 13, further including a demulcent in an amount ranging from about 0.05 to about 1% w/v.

19. The ophthalmic composition as in claim 13, further including a chelating agent in an amount ranging from about 0.001 to about 0.01% w/v.

20. The ophthalmic composition as in claim 13, further including a viscosity modifying agent in an amount ranging from about 0.01 to about 5% w/v.

21. An ophthalrnic composition, wherein the solution comprises:
from about 0.001% to about 1% w/v hyaluronic acid, from about 0.2 to 20 ppm cetylpyridinium chloride, and a buffer.

22. The ophthalmic composition as in claim 21, wherein the hyaluronic acid has a molecular weight of from about 70,000 to about 4 million Daltons.
